(19) United States
(12) Patent Application Publication
(10) Pub. No.: US 2011/0243905 A1
(43) Pub. Date: Oct. 6, 2011

(54) STRAIN OF HIGHLY MOSQUITOCIDAL BACILLUS

(76) Inventors: Hyun-Woo Park, Riverside, CA (US); Sabrina R. Hayes, Panama City, FL (US); Michael Hudon, Sebastian, FL (US)

(21) Appl. No.: 12/754,259

(22) Filed: Apr. 5, 2010

(51) Int. Cl. A01N 63/02; C12N 1/20; A01P 7/04

(52) U.S. Cl. 424/93.46; 435/252.5

(57) ABSTRACT

A new strain of Bacillus and a method and composition for controlling mosquitoes with the strain.

### FIELD OF THE INVENTION

The invention relates to a new strain of Bacillus and its use as a biopesticide. The new strain is highly mosquitocidal, particularly to the mosquito species Aedes taeniorhynchus, Culex quinquefasciatus, and Anopheles quadrimaculatus. The strain has been deposited at the Bacillus Genetic Stock Center and is identified as Bacillus Genetic Stock Center Accession Number PW2 (BGSC PW2).

### BACKGROUND OF THE INVENTION

Despite advances in medical science and new drugs, malaria, filariasis, dengue and the viral encephalitides remain important diseases of humans, with an estimated 2 billion people worldwide living in areas where these are endemic [The World Health Report—1999, World Health Organization, Geneva, Switzerland (1999)]. The causative agents of these diseases are transmitted by mosquitoes, and therefore disease control methods have relied heavily on broad spectrum chemical insecticides to reduce mosquito populations. However, chemical insecticide usage is being phased out in many countries due to the development of insecticide resistance in mosquito populations. Furthermore, many governments restrict use of these chemicals because of concerns over their effects on the environment, especially on non-target beneficial insects, and vertebrates through contamination of food and water supplies.

As a result of these problems, the World Health Organization is facilitating the replacement of chemical with bacterial insecticides through the development of standards for their registration and use [Guideline specifications for bacterial larvicides for public health use, WHO Document WHO/CDS/CPC/WHOPES/99.2, World Health Organization, Geneva, Switzerland (1999)]. Products based on bacteria have been designed to control mosquito larvae, and the two most widely used are VectoBac® and Teknar®, both of which are based on B. thuringiensis subsp. israelensis. In addition, VectoLex®, a new product based on B. sphaericus, has come to market recently for control of the mosquito vectors of filariasis and viral diseases. These products have achieved moderate commercial success, but their high cost and lower efficacy compared to many chemical pesticides prevents them from being used more extensively in many developing countries. Moreover, concerns have been raised about their long term utility due to resistance, which has already been reported to B. sphaericus in field populations of Culex mosquitoes in India, Brazil, and France (Singe, et al. First field occurrence of Culex pipiens resistance to Bs in southern France, VII European Meeting, Society for Vector Ecology, 5-8 September Barcelona, Spain (1994); Rao et al., J. Am. Mosq. Control Assoc. 11: 1-5 (1995); Silva-Filha et al., J Econ. Entomot 88: 525-530 (1995)).

The insecticidal properties of these bacteria are due primarily to insecticidal proteins produced during sporulation. In B. thuringiensis subsp. israelensis, the key proteins are Cyt1A (27 kDa), Cry11A (72 kDa), Cry4A (128 kDa) and Cry4B (134 kDa), whereas B. sphaericus produces 41- and 52-kDa proteins that serve, respectively, as the toxin and binding domains of a single binary toxin [Federici et al. in Bacterial Control of Mosquitoes and Blackflies, eds.: de Barjac & Sutherland, D. J, 11-44 (Rutgers University Press, New Brunswick, N.J.) (1990); Baumann et al., Microbiot Rev. 55:425-436 (1991)].

Over the past thirty years, many mosquitocidal strains of B. sphaericus have been isolated. The most toxic of these are strains 1593 and especially 2362, both of which belong to flagellar serotype 5a5b (Baumann et al, Bacillus sphaericus as a mosquito pathogen: properties of the organism and its toxins. Microbial Rev 55:425-436; 1991, Charles et al, Bacillus sphaericus toxins: molecular biology and mode of action. Annu Rev Entoniol 41:451-472; 1996). The principal toxin in these strains is a binary (Bin) toxin composed of two proteins, a 51.4-kDa binding domain (BinB) and 41.9-kDa toxin domain (BinA) that co-crystallize into a single small parasporal body. Strain 2362 has a median lethal concentration (LC₅₀) of 18 ng/ml against the fourth instar of Culex mosquitoes. A very similar binary toxin occurs in strain 2297 and forms a much larger parasporal body, but it is not as toxic (Berry et al, Nucleotide sequence of two toxin genes from Bacillus sphaericus IAB59: sequence comparisons between five highly toxinogenic strains. Nucleic Acids Res 17:7516, 1989; Genetic determinants of host ranges of Bacillus sphaericus mosquito larvicidal toxins. J Bacterial 175:510-518, 1993).

Other B. sphaericus strains produce other toxins known as Mtx (mosquitocidaltoxin) during vegetative growth. Three different types are known: Mtx1 (100 kDa), Mtx2 (31 kDa) and Mtx3 (36 kDa). Unlike the Bin toxin, they do not form crystals and, therefore, are degraded quickly upon their synthesis during the vegetative stage. Highly mosquitocidal strains such as 2297 and 2362 produce both types of toxins whereas others produce only either Bin or Mtx. After ingestion by mosquito larvae, the 51.4 and 41.9-kDa proteins are cleaved by proteases yielding peptides of 43 and 39 kDa, respectively, which form the active toxin. These associate, bind to a receptor on the midgut microvilli, and cause lysis of midgut cells after internalization [Davidson, Binding of the Bacillus sphaericus (Eubacteriales: Bacillaceae) toxin to midgut cells of mosquito (Diptera: Culicidae) larvae: relationship to host range. J Med Entomol 25:151-157, 1988].

Because of its longer persistence in polluted water and relatively high toxicity against certain species of mosquitoes, B. sphaericus 2362 (Weiser, “A mosquito-virulent Bacillus sphaericus in adult Simulium damnosum from northern Nigeria”, Zentralbl Mikrobiol 139:57-60, 1984), the most toxic strain known, has been marketed under the product name VectoLex® (Valent BioSciences) and used for mosquito control operations along with the other microbial insecticide, B. thuringiensis subsp. israelensis (Goldberg and Margalit, “A bacterial spore demonstrating rapid larvicidal activity against Anopheles sergentii, Uranotaenia unguiculata, Culex univitattus, Aedes aegypti and Culex pipiens, Mosq News 37:355-358, 1977).

The toxins produced by B. sphaericus are toxic to many species of mosquito larvae upon ingestion. All species of Culex larvae are susceptible to B. sphaericus. Many species of Aedes, Psorophora, Coquillettidia, Mansonia, and Anopheles are also very susceptible. However, susceptibility of species within these genera is variable.

A disadvantage of conventional B. sphaericus strains is that the binary toxin is in essence a single toxin, and significant levels of resistance, exceeding well over 20,000-fold, have already developed under operational field conditions where it has been used intensively (Yuan et al, High-level field resistance to Bacillus sphaericus C3-41 in Culex quinquefasciatus from southern China. Biocontr Sci Technol 10:41-49, 2000; Su and Mulla, Documentation of high-level Bacillus sphaericus 2362 resistance in field populations of Culex quinquefasciatus breeding in polluted water in Thailand. J Am Mosq Control Assoc 20:405-411, 2004). This resistance is due to a significant reduction in the binding of the B. sphaericus binary toxin to the microvillar membrane of resistant mosquitoes [Nielsen-LeRoux et al, Resistance in a laboratory population of Culex quinquefasciatus (Diptera: Culicidae)]. Mosquito resistance to Bs binary toxin is due to a change in the receptor on midgut brush-border membrane. (Eur J Biochem 228:206-210, 1995).

It is an object of the invention to provide a Bacillus strain that exhibits toxicity against mosquitoes. It is another object of the invention to provide a Bacillus strain that demonstrates improved efficacy compared to VectoLex®, as well as other biopesticides, against one or more mosquito variants.

### SUMMARY OF THE INVENTION

The above and other objects are realized by the present invention, which provides a new Bacillus strain VB24 that is highly mosquitocidal, particularly to the mosquito species Aedes taeniorhynchus, Culex quinquefasciatus, and Anopheles quadrimaculalus.

Additional embodiments of the invention concern methods for controlling mosquitoes comprising contacting a mosquito larva with a toxic amount of the Bacillus VB24 strain, or a crystal, polypeptide, protoxin, or toxin derived from the new strain.

Still more embodiments of the invention are compositions having toxic activity against mosquitoes comprising an effective amount of the above Bacillus VB24 strain, or a crystal, polypeptide, protoxin, or toxin derived from the new strain.

### BRIEF DESCRIPTION OF THE DRAWINGS

FIG. 1 is a schematic illustration of the phylogenetic relationships of Bacillus VB24.

FIG. 2 is an SDS-PAGE of a whole cell culture of Bacillus VB24. Lane M is molecular weight markers, lane 1 is another Bacillus, and lane 2 is Bacillus VB24.

FIG. 3 is a transmission electron micrograph of Bacillus VB24. S indicates spore.

### DETAILED DESCRIPTION OF THE INVENTION

The present invention is predicated on the discovery of a new strain of Bacillus and the discovery that the new strain exhibits high toxicity against several major species of mosquito larvae.

The new strain is termed Bacillus VB24 and is deposited at the Bacillus Genetic Stock Center and identified by Bacillus Genetic Stock Center Accession Number PW2 (BGSC PW2). The new strain has demonstrated particular toxicity against the mosquito species Aedes taeniorhynchus, Culex quinquefasciatus, and Anopheles quadrimaculatus.

Ae. taeniorhynchus is a severe biter of man and livestock along the southern coasts of the United States from North Carolina to Florida and in the Caribbean. Unchecked populations can have a major economic impact. While capable of transmitting eastern equine encephalitis and St. Louis encephalitis in the laboratory, it is not a major vector of these diseases in nature. It is, however, an important natural vector of dog heartworm and Venezuelan equine encephalitis.

Cx. quinquefasciatus is the major domestic pest in many urban areas, particularly as indicated by indoor biting. With respect to human disease it has been shown to be able to carry Murray Valley encephalitis (MVE) virus in laboratory studies. MVE virus has been isolated from the species in northern WA and it has yielded an isolate of Ross River (RR) virus during an outbreak in New Caledonia, but from a number of laboratory studies in Australia it appears to be a poor and unlikely vector of MVE, Kunjin, RR, and other arboviruses. It is a vector (not particularly efficient) of dog heartworm (and human filariasis in more northern tropical regions), an important vector of fowl pox, and possibly involved in myxomatosis transmission in some areas.

An. quadrimaculatus is the chief vector of malaria in North America. This species prefers habitats with well-developed beds of submergent, floating leaf, or emergent aquatic vegetation. Larvae are typically found in sites with abundant rooted aquatic vegetation, such as rice fields and adjacent irrigation ditches, freshwater marshes and the vegetated margins of lakes, ponds, and reservoirs.

Accordingly, it is evident that a biological pesticide having specific affinity for these mosquito species would be beneficial.

Isolation of the New Strain

The bacterium VB24 was isolated from a ditch in Indian River County, Florida using a method previously described by Park et al. [Park, H.-W. et al., 2007. Isolation of Bacillus sphaericus with improved efficacy against Culex quinquefasciatus. J. Am. Mosq. Control Assoc. 23: 478-480].

Mosquitocidal Assays of the New Strain

For preliminary mosquito bioassays Bacillus VB24 was grown in 50 ml of GYS medium for 3 days at 30° C., and Ochlerotatus (Aedes) taeniorhynchus and Cx. quinquefasciatus 4th instars maintained at John A. Mulrennan, Sr., Public Health Entomology Research & Education Center, Florida A & M University were used. Ten ml of undiluted bacterial culture was transferred to a 15 ml conical tube, 5 mosquito larvae were added, and tubes were placed at 28° C. Ten ml of tap water with 5 larvae was used as a control. Bacillus VB24 showed 100% mortality against these mosquito species after 24 h whereas the control did not show any mortality. Therefore, an advanced bioassay was performed to calculate LC₅₀s and to determine the host range of this isolate using a method previously established [Park, H.-W., D. K. Bideshi, and B. A. Federici. 2003. Recombinant strain of Bt producing Cyt1A, Cry11B, and the Bs binary toxin. Appl. Environ. Microbiol. 69: 1331-1334; Park, H.-W., D. K. Bideshi, M. C. Wirth, J. J. Johnson, W. E. Walton and B. A. Federici. 2005. Recombinant larvicidal bacteria with markedly improved efficacy against Culex vectors of West Nile Virus. Am. J. Trap. Med. Hyg. 72:732-738].

Lyophilized powder of sporulated cultures of Bacillus VB24 and B. thuringiensis subsp. israelensis 2362 (Institut Pasteur, Paris, France) was tested against Ae. aegypti, Cx. quinquefasciatus and Ochlerotatus (Aedes) taeniorhynchus 4th instars and An. quadrimaculatus 3rd instars. Ae. aegypti and An. quadrimaculatus eggs were purchased from Benzon Research (Carlisle, Pa.). The results showed that Bacillus VB24 did not show any toxicity against Ae. aegypti but it is highly toxic to An. quadrimaculatus (LC₅₀=6.0 ng/ml), Cx. quinquefasciatus (LC₅₀=6.7 ng/ml), and Ae. taeniorhynchus (LC₅₀=4.6 ng/ml). Bacillus VB24 showed higher toxicity against each of the mosquito species tested than B. sphaericus 2362. Table 1 illustrates the LC₅₀ (fiducial limits); 48 hour mortality in nanograms per milliliter; N/T means not able to measure LC₅₀ using 1 μg/ml as the highest concentration.

TABLE 1
Mosquitocidal activity of the new strain Bacillus VB24 compared to the known
B. sphaericus 2362.

| | Aedes | Anopheles | Culex | Aedes |
|---|---|---|---|---|
| Bacterial train | aegypti | quadrimaculatus | quinquefasciatus | taeniorhynchus |
| Bacillus VB24 | N/T | 6.0 (3.5-8.6) | 6.7 (4.5-8.9) | 4.6 (02.4-7.0) |
| B. sphaericus 2362 | N/T | 50.7 (37.8-70.1) | 12.6 (8.7-17.5) | N/T |

Characterization of the New Strain

GC-FAME and 16S rRNA Gene Sequence Alignment

For identification of Bacillus VB24, gas chromatographic analysis of fatty acid methyl esters (GC-FAME) and 16S rRNA gene sequence alignment were performed. For GC-FAME, recovered bacterium was grown on nutrient agar plates at 30° C. overnight and a 4 mm loop was used to harvest about 40 mg of bacterial cells of the streaked plates. The cells were placed in a clean 13×100 mm culture tubes. One nil of 15% NaOH in 50% aqueous methanol was added to each tube containing cells. The tubes were securely sealed with Teflon lined caps, vortexed briefly and heated in a boiling water bath for 5 min, at which time the tubes were vigorously vortexed for 10 sec and returned to the water bath to complete the 30 min heating. The cooled tubes were uncapped and 2 ml of methanolic HCl was added. The tubes were capped and briefly vortexed. After vortexing, the tubes were heated for 10 min at 80° C. Addition of 1.25 ml of hexane-methyl-tert-butyl ether (1:1; VAT) to the cooled tubes was followed by recapping and gentle tumbling on a rotary shaker for 10 min. The tubes were uncapped and the aqueous phase was pipetted out and discarded. About 3 ml of diluted NaOH was added to the organic phase remaining in the tubes, the tubes were recapped, and tumbled for 5 min.

Following uncapping, about ⅔ of the organic phase was pipetted into a gas chromatograph vial. Samples were analyzed with the 6850 Gas Chromatograph (Agilent technologies, Santa Clara, Calif.) and the Sherlock MIS software (MIDI Inc., Newark, Del.). For 16S rRNA gene sequence alignment, the 16S rRNA genes from Bacillus VB24 were amplified from genomic DNA by PCR. Primers used were universal 16S primers that correspond to positions 0005F and 0531R. Cycle sequencing of the 16S rRNA amplification products was carried out using DNA polymerase and dye terminator chemistry. Excess dye-labeled terminators were removed from the sequencing reactions, collected by centrifugation, dried under vacuum and frozen at −20° C. until ready to load. Samples were resuspended in a formamide solution and denatured prior to loading. The samples were electrophoresed on the 3130 Genetic Analyzer (Applied Biosystems, Foster City, Calif.) and analyzed using the DNA Library D16S2 (MIDI Inc., Newark, Del.).

Table 2 shows the D16S2 DNA match report and Table 3 shows the Cross Library Report.

TABLE 2
D16S2 DNA Match Report.

| Match | % Difference | Length | Library Entry Name |
|---|---|---|---|
| 1 | 2.23 | 536 | Bacillus fusiformis |
| 2 | 2.42 | 536 | Bacillus sphaericus |
| 3 | 7.01 | 536 | Bacillus insolitus |
| 4 | 8.03 | 536 | Bacillus firmus |
| 5 | 8.16 | 534 | Bacillus ureae |
| 6 | 8.53 | 536 | Bacillus pasteurii |
| 7 | 8.85 | 534 | Bacillus globispora |
| 8 | 9.03 | 534 | Bacillus psychrophila |
| 9 | 9.07 | 532 | Bacillus circulans |
| 10 | 9.17 | 534 | Bacillus lentus |

TABLE 3
Cross Library Report.

| % Difference | Genus | Species | FAME SI |
|---|---|---|---|
| 2.23 | Bacillus | fusiformis | 0.000 |
| 2.42 | Bacillus | sphaericus | 0.926 |
| 7.01 | Bacillus | insolitus | 0.000 |
| 8.03 | Bacillus | firmus | 0.000 |
| 8.16 | Bacillus | ureae | 0.000 |
| 8.53 | Bacillus | pasteurii | — |
| 8.85 | Bacillus | globispora | 0.000 |
| 9.03 | Bacillus | psychrophila | 0.000 |
| 9.07 | Bacillus | circulans | 0.000 |
| 9.17 | Bacillus | lentus | 0.000 |

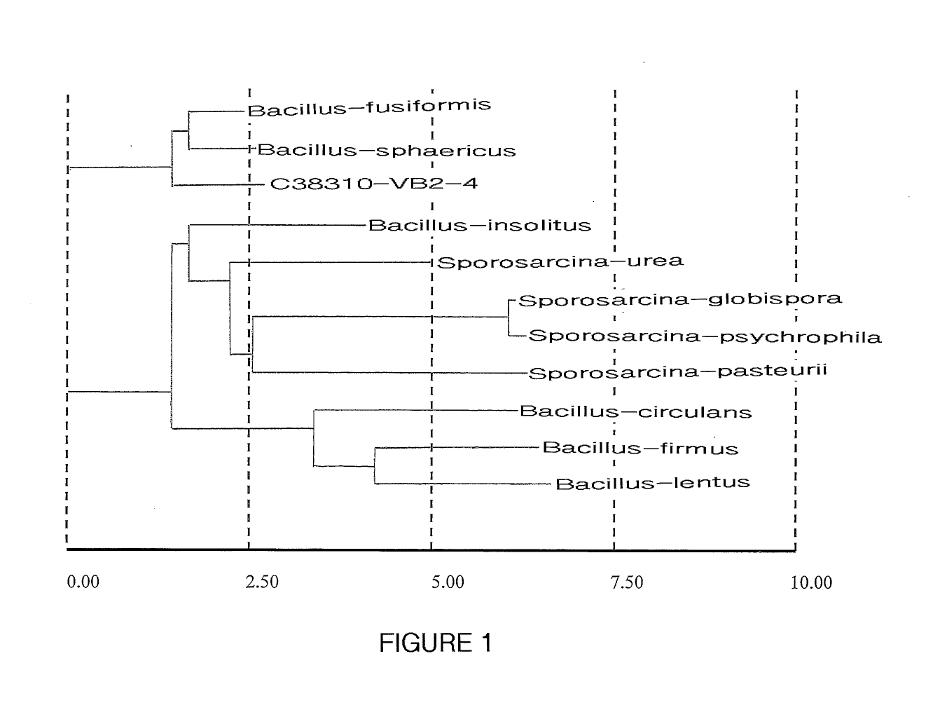

The results showed that Bacillus VB24 is closest in similarity to B. fusiformis and B. sphaericus. Bacillus VB24 has 2.23% genetic difference from B. fusiformis and 2.42% genetic difference from B. sphaericus and a Similarity Index of 0.926 to B. sphaericus FIG. 1 illustrates the phylogenetic neighbor joining tree for Bacillus VB24.

SDS-Polyacrylamide Gel Electrophoresis and Transmission Electron Microscopy

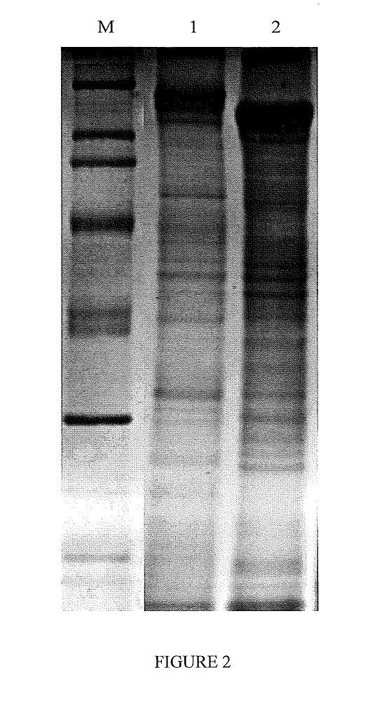

SDS-polyacrylamide gel electrophoresis (PAGE) was performed using the lysed GYS cultures of Bacillus VB24 according to established methods. The results are shown in FIG. 2. M indicates the MW markers, where in kDa and descending order, the major bands are 200, 116, 97, 66, 45, and 31. Lane 1 is another Bacillus and lane 2 is the new Bacillus VB24. The new strain has a different protein profile than the known B. sphaericus strains 2362 and 2297.

Transmission electron microscopy was performed on Bacillus VB24 as follows. Sporulated culture of Bacillus VB24 was processed using a laboratory microwave PELCO® BioWave with ColdSpot (Ted Pella, Inc., Redding, Calif.). Microwave settings were as follows: temperature probe in ColdSpot port, restricted temperature 37° C., vacuum 22 bars, 180 W unless otherwise stated. Cells were immersed into 2.5% glutaraldehyde in 0.1 M sodium cacodylate buffer, pH 7.2, once for 45 sec, under vacuum, buffer washed three times for 45 sec, post-fixed with 1% buffered OsO4 under vacuum, 1 min at room temperature, heated for 45 sec using a microwave, 3 min at room temperature, water washed twice for 45 see, and dehydrated in a graded ethanol series once for 45 sec followed by 100% acetone twice for 45 sec. Dehydrated samples were then infiltrated in a graded acetone/Spurrs resin series, 250 W, for 3 min under vacuum and embedded in 100% Spurn resin (1), polymerized in laboratory oven at 60° C., 2 days. Cured sample resin blocks were trimmed with Leica EM Trim (Leica Microsystems, Inc., Bannockburn, Ill.) sections were cut with a Leica UltraCut R, and ultrathin sections were collected on 200 mesh Cu grids.

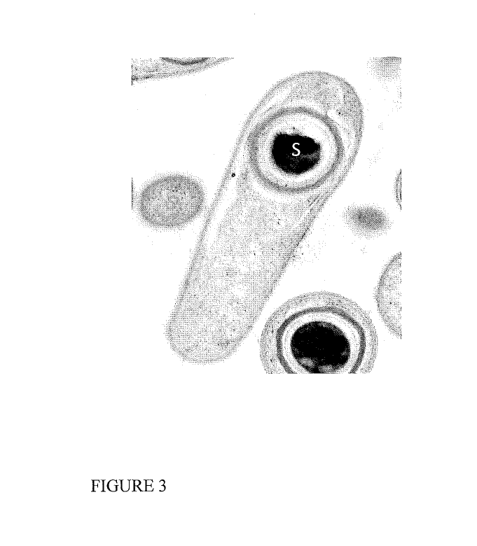

Ultrathin sections were post-stained with 2% aqueous uranyl acetate and Reynold's lead citrate, examined in a Hitachi H-7000 (Hitachi High Technologies America, Inc., Pleasanton, Calif.) operated at 75 kV and digital images acquired with MegaViewIII camera (Soft Imaging Solutions Corp, Lakewood, Colo.). FIG. 3 shows the bacterium fully sporulated and without an inclusion body.

Insecticidal Compositions

The Bacillus VB24 strain, its crystals, crystal proteins, protoxins, and/or its toxins can be used as the active ingredient in an insecticide composition used to control mosquitoes. For example, the Bacillus VB24 crystals can be isolated from sporulated cultures of the Bacillus VB24 strain and then the protoxins and mixtures can be isolated from these crystals. An insecticide composition can be formulated in a conventional manner using the Bacillus VB24 strain or its crystals, crystal proteins, protoxins, and/or toxins, together with suitable carriers, diluents, emulsifiers, and/or dispersants. This insecticide composition can be formulated as a wettable powder, pellets, granules, or dust, or as a liquid formulation with aqueous or non-aqueous solvents as a foam, gel, suspension, concentrate, etc. The concentration of the Bacillus VB24, crystals, crystal proteins, protoxins or toxins in such a composition will depend upon the nature of the formulation and its intended mode of use. Generally, an insecticide composition of this invention can be used to protect an area for one day to a few weeks, with each application of the composition. For more extended protection additional amounts of the composition would have to be applied periodically.

A method for controlling mosquitoes in accordance with this invention preferably comprises applying to the locus (area) to be protected an insecticidal amount of the Bacillus VB24 strain or its crystals, crystal proteins, protoxins, and/or toxins in an insecticidal composition as described above.

The examples in this application serve to further illustrate the invention, to provide those of ordinary skill in the art with a complete disclosure and description of how the compounds, compositions, articles, devices, and/or methods claimed herein are made and evaluated, and are not intended to limit the scope of the invention.

Modifications and variations of the present invention will be apparent to those skilled in the art from the forgoing detailed description. AU modifications and variations are intended to be encompassed by the following claims. All publications, patents, and patent applications cited herein are hereby incorporated by reference in their entirety.

What is claimed is:

1. A Bacillus strain identified by Bacillus Genetic Stock Center Accession Number PW2 (BGSC PW2).

2. A method for controlling mosquitoes comprising contacting a mosquito larva with a toxic amount of the Bacillus strain of claim 1, or its crystals, crystal proteins, protoxins, or toxins.

3. A composition having toxic activity against mosquitoes comprising an effective amount of the Bacillus strain of claim 1, or its crystals, crystal proteins, protoxins, or toxins.

4. A Bacillus strain that has greater activity against the mosquito species Aedes taeniorhynchus, Culex quinquefasciatus, and Anopheles quadrimaculatus than B. sphaericus 2362.
